(19) United States
(12) Patent Application Publication
(10) Pub. No.: US 2015/0342540 A1
(43) Pub. Date: Dec. 3, 2015

(54) HEART FAILURE EVENT DETECTION AND RISK STRATIFICATION USING HEART RATE TREND

(71) Applicant: Cardiac Pacemakers, Inc., St. Paul, MN (US)

(72) Inventors: Qi An, Blaine, MN (US); Yi Zhang, Plymouth, MN (US); Pramodsingh Hirasingh Thakur, Woodbury, MN (US); Viktoria A. Averina, Roseville, MN (US)

(21) Appl. No.: 14/684,978

(22) Filed: Apr. 13, 2015

Related U.S. Application Data
(60) Provisional application No. 62/005,548, filed on May 30, 2014.

(51) Int. Cl. A61B 5/00; A61B 5/11; A61B 5/0402; A61B 5/0205

(52) U.S. Cl. CPC A61B 5/7282; A61B 5/4857; A61B 5/7246; A61B 5/4809; A61B 5/02055; A61B 5/1116; A61B 5/4866; A61B 5/1118; A61B 5/7275; A61B 5/7264; A61B 5/7278; A61B 5/686; A61B 5/0006; A61B 5/0402; A61B 5/02405

(57) ABSTRACT

Systems and methods for detecting heart failure (HF) events or identifying patient at elevated risk of developing future HF events such as HF decompensation are described. A medical system can detect a contextual condition associated with a patient, including an environmental context or a physiologic context. The contextual condition includes information indicative or correlative of a change in metabolic demand. The system can include a heart rate (HR) analyzer circuit that extracts a HR feature from a cardiac activity signal, and perform multiple HR feature measurements in response to the detected patient contextual condition meeting a specified criterion. The system can calculate one or more signal metrics including a HR metric using the HR feature measurements. The system can detect an HF event using the signal metrics, or use the signal metrics to calculate a composite risk indicator indicative of the patient's likelihood of developing a future HF event.

### CLAIM OF PRIORITY

This application claims the benefit of priority under 35 U.S.C. §119(e) of U.S. Provisional Patent Application Ser. No. 62/005,548, filed on May 30, 2014, which is herein incorporated by reference in its entirety.

### TECHNICAL FIELD

This document relates generally to medical devices, and more particularly, to systems, devices and methods for detecting and monitoring heart failure decompensation.

### BACKGROUND

Congestive heart failure (CHF) is a major health problem and affects over five million people in the United States alone. CHF is the loss of pumping power of the heart, resulting in the inability to deliver enough blood to meet the demands of peripheral tissues. CHF patients typically have enlarged heart with weakened cardiac muscles, resulting in reduced contractility and poor cardiac output of blood.

CHF is usually a chronic condition, but can occur suddenly. It can affect the left heart, right heart or both sides of the heart. If CHF affects the left ventricle, signals that control the left ventricular contraction are delayed, and the left and right ventricles do not contract simultaneously. Non-simultaneous contractions of the left and right ventricles further decrease the pumping efficiency of the heart.

### OVERVIEW

Frequent monitoring of CHF patients and timely detection of events indicative of heart failure (HF) decompensation status can help prevent worsening HF in CHF patients, hence reducing cost associated with HF hospitalization. Additionally, identification of patient at an elevated risk of developing future HF events such as worsening HF can help ensure timely treatment, thereby improving the prognosis and patient outcome. Identifying and safely managing the patients having risk of future HF events can avoid unnecessary medical intervention and reduce healthcare cost.

Ambulatory medical devices or other signal analyzer circuits can be used for monitoring HF patient and detecting HF decompensation events. Examples of such ambulatory medical devices or signal analyzer circuits can include implantable medical devices (IMD), subcutaneous medical devices, wearable medical devices or other external medical devices. The ambulatory or implantable medical devices can include physiologic sensors which can be configured to sense electrical activity and mechanical function of the heart, or physical or physiological variables associated with the signs and symptoms of worsening HF. The medical device can optionally deliver therapy such as electrical stimulation pulses to a target area, such as to restore or improve the cardiac function or neural function. Some of these devices can provide diagnostic features, such as using transthoracic impedance or other sensor signals. For example, fluid accumulation in the lungs decreases the transthoracic impedance due to the lower resistivity of the fluid than air in the lungs. Fluid accumulation in the lungs can also irritate the pulmonary system and leads to decrease in tidal volume and increase in respiratory rate.

Some ambulatory medical devices can include a physiologic sensor configured to detect cardiac electrical or mechanical activities. Other physiologic sensors can be configured to detect heart sounds, cardiac electrical activity, body chemical activity, or other physiologic information. Such information can be used to provide a CHF event predictor metric to aid in diagnostic or therapeutic subject treatment. The present inventors have recognized, among other things, that information about a change in a subject's heart rate can be used to provide a CHF event predictor, or a risk stratifier predicting the likelihood the subject developing a future event indicative of CHF. For example, the present inventors have recognized that heart rate measured under a specified contextual condition, such as during a particular time of day or during a period of specified physical activity or exertion level, can provide an indication of worsening CHF. The indication of worsening CHF can be used by a clinician, or used automatically by a device, to update a subject therapy, such as to attempt to avert the predicted CHF event by addressing one or more underlying physiologic conditions. Various embodiments described herein can help improve detection of an HF event indicative of worsening HF, or improve process of identifying patients at elevated risk of developing future HF events.

Example 1 can include a system that can detect an HF event or predict the risk of HF event using one or more signal metrics generated from physiologic signals. The system can include a context detector circuit that can detect contextual condition associated with a patient, such as an environmental context or a physiologic context of a patient. The contextual condition can include information indicative or correlative of changes in metabolic demand of the patient, such as an elevated metabolic demand. A heart rate analyzer circuit can sense a cardiac activity signal, extract a HR feature from the sensed cardiac activity signal, and perform a plurality of measurements of the HR feature in response to the detected patient contextual condition meeting a specified criterion. The system can include a physiologic event detector circuit coupled to the target event indicator generator circuit. The physiologic event detector circuit can detect a target physiologic event using the one or more signal metrics. Additionally, or alternatively, the system can include a risk stratifier circuit that can calculate a composite risk indicator indicative of the likelihood of the patient developing a future event indicative of worsening HF.

Example 2 can include, or can optionally be combined with the subject matter of Example 1 to optionally include a target event indicator generator circuit that can calculate the one or more signal metrics including a statistical or morphological parameter extracted from the plurality of measurements of the HR feature, and a physiologic event detector circuit that can detect the worsening heart failure (HF) using the one or more signal metrics.

Example 3 can include, or can optionally be combined with the subject matter of one or any combination of Examples 1 and 2 to optionally include a target event indicator generator circuit that can calculate the one or more signal metrics including a heart rate (HR) metric, and a physiologic event detector circuit that can detect a target physiologic event in response to the HR metric exceeding a HR threshold.

Example 4 can include, or can optionally be combined with the subject matter of one or any combination of Examples 1 through 3 to optionally include a target event indicator generator circuit that can calculate the one or more signal metrics including a central tendency of the plurality of measurements of the HR feature.

Example 5 can include, or can optionally be combined with the subject matter of one or any combination of Examples 1 through 4 to optionally include a target event indicator generator circuit that can calculate the one or more signal metrics including a representative HR (HR_{Rep}) representing a specified percentile rank among the plurality of measurements of the HR feature, the specified percentile rank indicative of a relative number of HR measurements falling below or equal to the HR_{Rep}. In an example, the specified percentile rank includes a HR percentile greater than 50-th percentile, or a HR percentile of 85-th percentile.

Example 6 can include, or can optionally be combined with the subject matter of one or any combination of Examples 1 through 5 to optionally include a cardiac activity sensor coupled to the HR analyzer circuit, where the cardiac activity sensor can sense a cardiac electrical signal or a cardiac mechanical signal.

Example 7 can include, or can optionally be combined with the subject matter of one or any combination of Examples 1 through 6 to optionally include a timer/clock circuit capable of determining time of a day, and a HR analyzer circuit that can perform a plurality of measurements of the HR feature during specified time of a day indicative or correlative of an elevated metabolic demand of the patient.

Example 8 can include, or can optionally be combined with the subject matter of one or any combination of Examples 1 through 7 to optionally include a HR analyzer circuit that can perform a plurality of measurements of the HR feature during a period of time excluding night of the day.

Example 9 can include, or can optionally be combined with the subject matter of one or any combination of Examples 1 through 6 to optionally include a sleep state detector configured to detect in the patient a time of transition from a sleep state to an awake state, and a HR analyzer circuit that can perform a plurality of measurements of the HR feature in response to the detected transition from the sleep state to the awake state.

Example 10 can include, or can optionally be combined with the subject matter of one or any combination of Examples 1 through 6 to optionally include a posture sensor that can detect a posture of the patient and to classify the posture as one of two or more posture states, and a HR analyzer circuit that can perform a plurality of measurements of the HR feature in response to the detected posture being classified as a specified state indicative or correlative of an elevated metabolic demand.

Example 11 can include, or can optionally be combined with the subject matter of one or any combination of Examples 1 through 6 to optionally include one or more physiologic sensors that can detect a change in metabolic demand of the patient during a specified period, and a HR analyzer circuit that can perform a plurality of measurements of the HR feature in response to a detection of an increase in the metabolic demand.

Example 12 can include, or can optionally be combined with the subject matter of one or any combination of Examples 1 through 6 to optionally include an activity sensor that can detect a physical activity or exertion level of the patient, and a HR analyzer circuit that can perform a plurality of measurements of the HR feature in response to the detected physical activity or exertion level exceeding a specified activity level threshold.

Example 13 can include, or can optionally be combined with the subject matter of one or any combination of Examples 1 through 12 to optionally include an HR analyzer circuit that can receive a cardiac activity signal from the patient, extract a HR feature from the sensed cardiac activity signal, and perform the plurality of measurements of the HR feature in response to the received time of day meets a specified criterion or the received physical activity or exertion level exceeds a specified threshold value, and a physiologic event detector that can generate a composite risk indicator (CRI) using the one or more signal metrics, the CRI indicative of the likelihood of the patient developing a future event indicative of a new disease or worsening an existing disease.

Example 14 can include, or can optionally be combined with the subject matter of Example 13 to optionally include a physiologic event detector that can generate two or more categorical risk levels using a comparison between the composite risk indicator and a reference measure, the two or more categorical risk levels indicative of elevated risk of the patient developing a future event indicative of worsening heart failure.

Example 15 can include a method for detecting from a patient a contextual condition including environmental or physiologic context. The method includes sensing a cardiac activity signal from the patient and extracting a HR feature using the sensed cardiac activity signal. The method includes measuring a plurality of measurements of the heart rate features in response to the detected patient contextual condition meeting a specified criterion, and calculating one or more signal metrics using the measurements of the heart rate features. The signal metrics can include statistical features or morphological features extracted from a heart rate trend. The method can further include using the signal metrics to detect a target physiologic event indicative of worsening HF, or to generate a composite risk indicator that can predict the risk of the patient developing a future event indicative of worsening HR.

Example 16 can include, or can optionally be combined with the subject matter of Example 15 to optionally include calculating a statistical or morphological parameter extracted from the plurality of measurements of the HR feature.

Example 17 can include, or can optionally be combined with the subject matter of one or any combination of Examples 15 and 16 to optionally include calculating a representative HR (HR_{Rep}) representing an n-th percentile rank indicative of a relative amount of HR measurements among the plurality of measurements falling below or equal to the HR_{Rep}, the n-th percentile rank higher than 50-th percentile.

Example 18 can include, or can optionally be combined with the subject matter of one or any combination of Examples 15 and 17 to optionally include detecting time of a day, and measuring a plurality of measurements of the HR feature during specified time of a day indicative or correlative of an elevated metabolic demand of the patient.

Example 19 can include, or can optionally be combined with the subject matter of one or any combination of Examples 15 and 17 to optionally include detecting a physical activity or exertion level of the patient, and measuring a plurality of measurements of the HR feature in response to the detected physical activity or exertion level exceeding a specified activity level threshold.

Example 20 can include, or can optionally be combined with the subject matter of one or any combination of Examples 15 and 17 to optionally include using one or more physiologic sensors to detect at least one of a time of transition from a sleep state to an awake state, an increase in body temperature, an increase in heart rate, an increase in pressure, a decrease in physical activity or exertion level, or an increase in respiration rate.

Example 21 can include, or can optionally be combined with the subject matter of one or any combination of Examples 15 and 20 to optionally include detecting a target physiologic event indicative of worsening heart failure using the one or more signal metrics, or generating a composite risk indicator using the selected one or more signal metrics and classifying the patient into one of two or more categorical risk levels, the composite risk indicator indicative of the likelihood of the patient developing a future event indicative of worsening heart failure.

In the claims, the term “physiologic event detector” may be understood to cover the meaning “risk stratifier” in this sense of the word, a particular likelihood of the patient developing a future event indicative of a new disease or worsening an existing disease being understood as a target physiologic event of a particular type.

This Overview is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates an example of a cardiac rhythm management (CRM) system and portions of the environment in which the CRM system operates.

FIG. 2 illustrates an example of a heart rate trend-based physiologic event detector circuit.

FIG. 3 illustrates an example of a heart rate-based heart failure risk stratifier circuit.

FIG. 4 illustrates an example of a context receiver circuit.

FIG. 5 illustrates an example of a heart failure risk stratifier circuit.

FIG. 6 illustrates an example of a heart rate signal metric generator circuit.

FIG. 7 illustrates an example of a method for evaluating heart rate under specified contextual conditions.

FIG. 8 illustrates an example of a method for detecting a heart failure event indicative of heart failure decompensation or providing a risk stratification of a future heart failure event.

### DETAILED DESCRIPTION

Disclosed herein are systems, devices, and methods for detecting an event indicative of worsening heart failure (HF) such as an HF decompensation event, and/or for identifying patients with elevated risk of developing future events related to worsening HF. The HF event detection or HF risk stratification can be performed using the physiologic signals such as sensed from a physiologic sensor associated with an ambulatory medical device such as an implantable cardiac device. The present inventors have recognized that contextual conditions, including ambient environmental contexts and patient physiologic contexts, can affect certain types of sensor signals in HF patients including a heart rate. Therefore, by selectively acquiring sensor signals according to the patient contextual conditions and analyzing the signal metrics derived from the selectively acquired sensor signals, the present document can provide a method and device to detect the HF event indicative of worsening HF, or to predict the risk of future HF event, thereby allowing immediate medical attention to the patient.

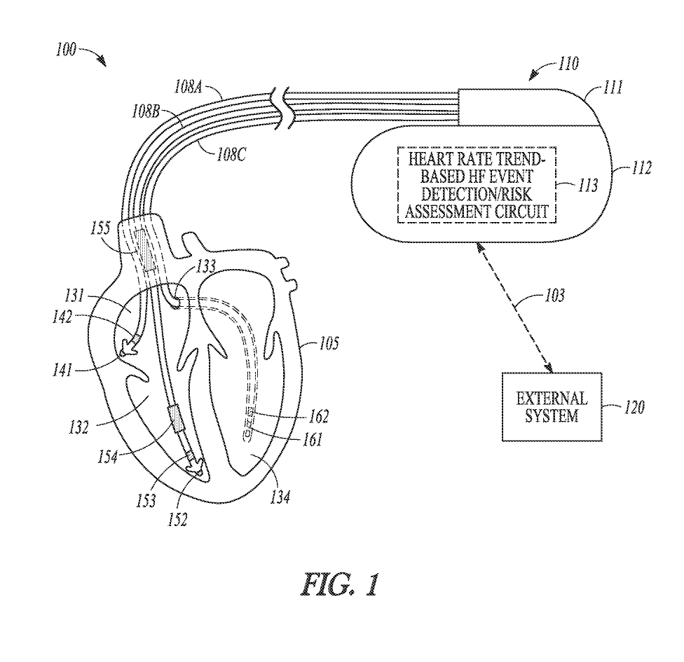

FIG. 1 illustrates an example of a Cardiac Rhythm Management (CRM) system 100 and portions of an environment in which the CRM system 100 can operate. The CRM system 100 can include an ambulatory medical device, such as an implantable medical device (IMD) 110 that can be electrically coupled to a heart 105 such as through one or more leads 108A-C, and an external system 120 that can communicate with the IMD 110 such as via a communication link 103. The IMD 110 may include an implantable cardiac device such as a pacemaker, an implantable cardioverter-defibrillator (ICD), or a cardiac resynchronization therapy defibrillator (CRT-D). The IMD 110 can include one or more monitoring or therapeutic devices such as a subcutaneously implanted device, a wearable external device, a neural stimulator, a drug delivery device, a biological therapy device, a diagnostic device, or one or more other ambulatory medical devices. The IMD 110 may be coupled to, or may be substituted by a monitoring medical device such as a bedside or other external monitor.

As illustrated in FIG. 1, the IMD 110 can include a hermetically sealed can 112 that can house an electronic circuit that can sense a physiological signal in the heart 105 and can deliver one or more therapeutic electrical pulses to a target region, such as in the heart, such as through one or more leads 108A-C. The CRM system 100 can include only one lead such as 108B, or can include two leads such as 108A and 108B.

The lead 108A can include a proximal end that can be configured to be connected to IMD 110 and a distal end that can be configured to be placed at a target location such as in the right atrium (RA) 131 of the heart 105. The lead 108A can have a first pacing-sensing electrode 141 that can be located at or near its distal end, and a second pacing-sensing electrode 142 that can be located at or near the electrode 141. The electrodes 141 and 142 can be electrically connected to the IMD 110 such as via separate conductors in the lead 108A, such as to allow for sensing of the right atrial activity and optional delivery of atrial pacing pulses. The lead 108B can be a defibrillation lead that can include a proximal end that can be connected to IMD 110 and a distal end that can be placed at a target location such as in the right ventricle (RV) 132 of heart 105. The lead 108B can have a first pacing-sensing electrode 152 that can be located at distal end, a second pacing-sensing electrode 153 that can be located near the electrode 152, a first defibrillation coil electrode 154 that can be located near the electrode 153, and a second defibrillation coil electrode 155 that can be located at a distance from the distal end such as for superior vena cava (SVC) placement. The electrodes 152 through 155 can be electrically connected to the IMD 110 such as via separate conductors in the lead 108B. The electrodes 152 and 153 can allow for sensing of a ventricular electrogram and can optionally allow delivery of one or more ventricular pacing pulses, and electrodes 154 and 155 can allow for delivery of one or more ventricular cardioversion/defibrillation pulses. In an example, the lead 108B can include only three electrodes 152, 154 and 155. The electrodes 152 and 154 can be used for sensing or delivery of one or more ventricular pacing pulses, and the electrodes 154 and 155 can be used for delivery of one or more ventricular cardioversion or defibrillation pulses. The lead 108C can include a proximal end that can be connected to the IMD 110 and a distal end that can be configured to be placed at a target location such as in a left ventricle (LV) 134 of the heart 105. The lead 108C may be implanted through the coronary sinus 133 and may be placed in a coronary vein over the LV such as to allow for delivery of one or more pacing pulses to the LV. The lead 108C can include an electrode 161 that can be located at a distal end of the lead 108C and another electrode 162 that can be located near the electrode 161. The electrodes 161 and 162 can be electrically connected to the IMD 110 such as via separate conductors in the lead 108C such as to allow for sensing of the LV electrogram and optionally allow delivery of one or more resynchronization pacing pulses from the LV.

The IMD 110 can include an electronic circuit that can sense a physiological signal. The physiological signal can include an electrogram or a signal representing mechanical function of the heart 105. The hermetically sealed can 112 may function as an electrode such as for sensing or pulse delivery. For example, an electrode from one or more of the leads 108A-C may be used together with the can 112 such as for unipolar sensing of an electrogram or for delivering one or more pacing pulses. A defibrillation electrode from the lead 108B may be used together with the can 112 such as for delivering one or more cardioversion/defibrillation pulses. In an example, the IMD 110 can sense impedance such as between electrodes located on one or more of the leads 108A-C or the can 112. The IMD 110 can be configured to inject current between a pair of electrodes, sense the resultant voltage between the same or different pair of electrodes, and determine impedance using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing. In an example, the IMD 110 can be configured to inject current between an electrode on the RV lead 108B and the can housing 112, and to sense the resultant voltage between the same electrodes or between a different electrode on the RV lead 108B and the can housing 112. A physiologic signal can be sensed from one or more physiological sensors that can be integrated within the IMD 110. The IMD 110 can also be configured to sense a physiological signal from one or more external physiologic sensors or one or more external electrodes that can be coupled to the IMD 110. Examples of the physiological signal can include one or more of electrocardiogram, intracardiac electrogram, arrhythmia, heart rate, heart rate variability, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physical activity or exertion level, physiologic response to activity, posture, respiration, body weight, or body temperature.

The arrangement and functions of these leads and electrodes are described above by way of example and not by way of limitation. Depending on the need of the patient and the capability of the implantable device, other arrangements and uses of these leads and electrodes are possible.

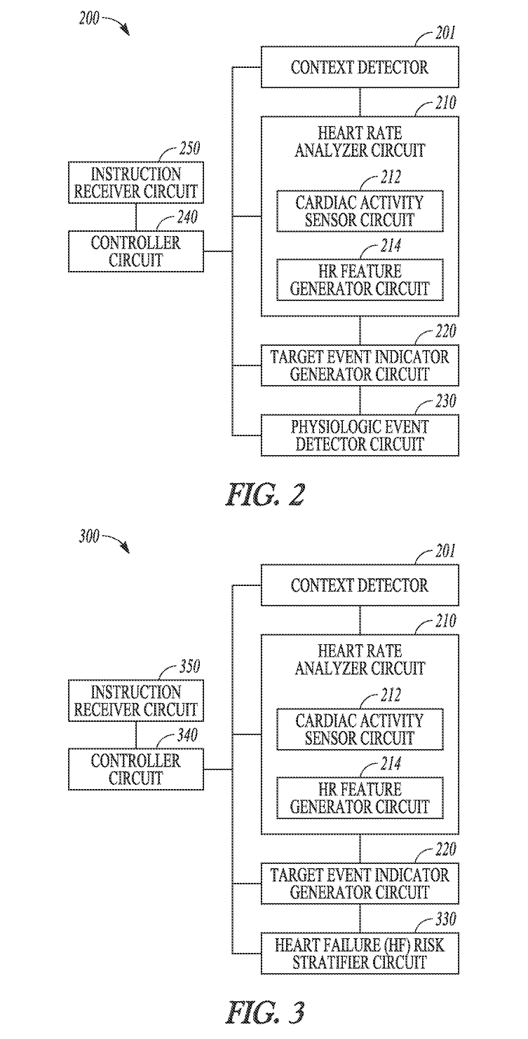

As illustrated, the CRM system 100 can include a heart rate trend-based HF event detection/risk assessment circuit 113. The heart rate trend-based HF event detection/risk assessment circuit 113 can receive a cardiac activity signal such as an electrocardiogram (ECG) or an intracardiac electrogram (EGM), each indicative of electrical activity of the heart. The EGM can be sensed using ambulatory physiologic sensors deployed on or within the patient and communicated with the IMD 110, such as electrodes on one or more of the leads 108A-C and the can 112, or ambulatory physiologic sensors deployed on or within the patient and communicated with the IMD 110. The heart rate trend-based HE event detection/risk assessment circuit 113 can calculate a heart rate feature using the cardiac activity signal when a contextual condition meets a specified criterion. The heart rate trend-based HF event detection/risk assessment circuit 113 can use at least the heart rate feature to generate a target event indicator such as a risk indicator indicative of a likelihood of the patient developing a future HF decompensation event. The HF decompensation event can include one or more early precursors of an HF decompensation episode, or an event indicative of HF progression such as recovery or worsening HF status. Examples of the heart rate trend-based HF event detection/risk assessment circuit 113 are described below, such as with reference to FIGS. 2-6.

The external system 120 can allow for programming of the IMD 110 and can receive information about one or more signals acquired by IMD 110, such as can be received via a communication link 103. The external system 120 can include a local external IMD programmer. The external system 120 can include a remote patient management system that can monitor patient status or adjust one or more therapies such as from a remote location.

The communication link 103 can include one or more of an inductive telemetry link, a radio-frequency telemetry link, or a telecommunication link, such as an interact connection. The communication link 103 can provide for data transmission between the IMD 110 and the external system 120. The transmitted data can include, for example, real-time physiological data acquired by the IMD 110, physiological data acquired by and stored in the IMD 110, therapy history data or data indicating IMD operational status stored in the IMD 110, one or more programming instructions to the IMD 110 such as to configure the IMD 110 to perform one or more actions that can include physiological data acquisition such as using programmably specifiable sensing electrodes and configuration, device self-diagnostic test, or delivery of one or more therapies.

The heart rate trend-based HF event detection/risk assessment circuit 113 may be implemented at the external system 120, which can be configured to perform HF risk stratification HF event detection, such as using data extracted from the IMD 110 or data stored in a memory within the external system 120. Portions of heart rate trend-based HF event detection/risk assessment circuit 113 may be distributed between the IMD 110 and the external system 120.

Portions of the IMD 110 or the external system 120 can be implemented using hardware, software, or any combination of hardware and software. Portions of the IMD 110 or the external system 120 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. While described with reference to the IMD 110, the CRM system 100 could include subcutaneous medical device (e.g., subcutaneous ICD, subcutaneous diagnostic device), wearable medical devices (e.g., patch based sensing device), or other external medical devices.

FIG. 2 illustrates an example of a heart rate trend-based physiologic event detector circuit 200, which can be an embodiment of the heart rate trend-based HF event detection/risk assessment circuit 113. The heart rate trend-based physiologic event detector circuit 200 can also be implemented in an external system such as a patient monitor configured for providing the patient's diagnostic information to an end-user. The heart rate trend-based physiologic event detector circuit 200 can include one or more of a context detector 201, a heart rate analyzer circuit 210, a target event indicator generator circuit 220, a physiologic event detector circuit 230, a controller circuit 240, and an instruction receiver circuit 250.

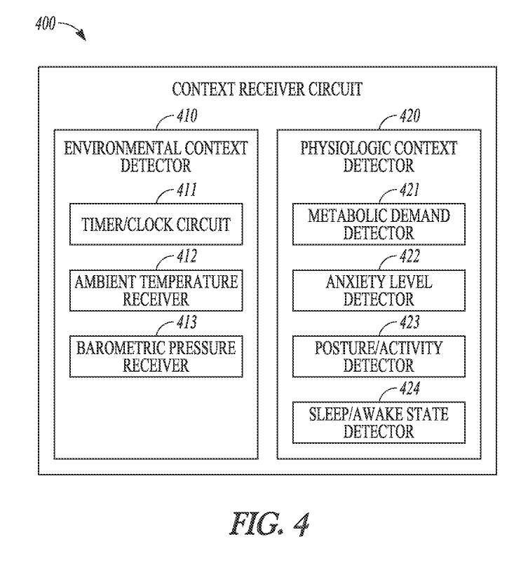

The context detector 201 can be configured to detect contextual condition associated with a patient. The contextual condition can include a patient's physiologic context or an environmental context. The physiologic context includes body-related contextual information, such as the posture, physical activity or exertion level, sleep or awake state, mental or emotional state, metabolic demand, body temperature, patient weight, body fluid status, and other parameters indicative of the patient's body conditions or health status. The environmental context can include factors external to the patient but likely to affect patient's health or disease states. Examples of the environmental context can include ambient temperature, barometric pressure, humidity, or social environment. The physiologic context or the environment context can correlate to or be indicative of a change in the patient's metabolic demand such as elevated metabolic demand. Examples of the context detector 201 are discussed below, such as with reference to FIG. 4.

The heart rate analyzer circuit 210 can include a cardiac activity sensor circuit 212 and an HR feature generator circuit 214. The cardiac activity sensor circuit 212 can include one or more implantable, wearable, or otherwise ambulatory cardiac activity sensors each configured to sense from a patient a cardiac activity signal. In an example, the cardiac activity signal can be a cardiac electrical signal. The cardiac activity sensor circuit 212 can be coupled to two or more electrodes on one or more of the leads 108A-C or the can 112, and sense an electrogram (EGM) from inside the heart chamber, at or within the heart tissue, or on or near the surface of the heart. The electrodes can be placed subcutaneously (e.g., under the skin) to sense a subcutaneous electrocardiogram (ECG) signal. The electrodes can also be non-invasively attached to the body skin to sense a surface ECG signal.

Alternatively or additionally, the cardiac activity sensor circuit 212 can be configured to sense cardiac mechanical activity indicative or correlative of contractions of the heart. In an example, the cardiac activity sensor circuit 212 can include an accelerometer or a microphone configured to sense a heart sound signal. In another example, the cardiac activity sensor circuit 212 can include an impedance sensor configured to sense intracardiac impedance change as a result of cyclic cardiac contractions. The cardiac activity sensor circuit 212 can also be configured to sense a physiologic activity caused by cardiac contractions. For example, a blood pressure or blood flow sensor can be used to sense arterial pulse signal indicative of pulsatile blood pressure or blood flow caused by cyclic contraction of the heart and the opening/closure of aortic or pulmonic valves.

The cardiac activity sensor circuit 212 can include sub-circuits to process the sensed cardiac activity signals, including signal amplification, digitization, filtering, or other signal conditioning processes. The cardiac activity sensor circuit 212 can detect from the processed cardiac electrical signal electrophysiological events such as events indicative of depolarization, hyperpolarization, or repolarization of a specified portion of the heart, such as an atrium or a ventricle. Examples of the electrophysiological events can include a P wave, an R wave, a QRS complex, a T wave, or other ECG components representing electrophysiological activities of the myocardium. The electrophysiological events can also include atrial sensed events or ventricular sensed events from an EGM. Additionally or alternatively, the cardiac activity sensor circuit can detect from the cardiac mechanical signal mechano-physiologic events indicative of a specified state during a cardiac contraction cycle such as atrial contraction, ventricular contraction, end of emptying, or end of filling, among others. Examples of the mechano-physiologic events can include S1, S2, S3, or S4 heart sound from the sensed heart sound signal, peak or trough impedance from the cardiac impedance signal, or peak or trough blood pressure from the blood pressure signal, among others.

In some examples, the cardiac electrical or cardiac mechanical signals can be acquired from a patient and stored in a storage device such as an electronic medical record (EMR) system. The heart rate analyzer circuit 210 can be coupled to the storage device and retrieve from the storage device one or more cardiac electrical or cardiac mechanical signals in response to a command signal. The command signal can be issued by an end-user such as via an input device coupled to the instruction receiver 250, or generated automatically in response to a specified event.

The HR feature generator circuit 214 can be configured to generate one or more HR features using the detected electrophysiological events or the detected mechano-physiologic events. In an example, the HR feature can include a cardiac cycle length (CL) or a heart rate derived from the cardiac cycle (e.g., HR=60/CL). The CL can be measured using timing of one or more of the detected electrophysiological events such as intervals between two adjacent R waves (R-R interval) or two adjacent P waves (P-P interval), or intervals between two adjacent impedance peaks from the cardiac impedance signal or between two adjacent blood pressure peaks (systolic pressure) or two adjacent blood pressure troughs (diastolic pressure). In another example, the HR feature can include a variability of HR or a variability of CL, such as beat-to-beat difference in HR or CL, or short-term variance or standard deviation of HR or CL.

As illustrated in FIG. 2, the heart rate analyzer circuit 210 can be coupled to the context detector 201 and receive the detected contextual conditions such as patient physiologic context or environmental context information. The HR feature generator circuit 214 can perform a plurality of HR or CL measurements in response to the detected contextual condition meeting a specified criterion. The patient physiologic context information, or the ambient environment context, may affect the quality of the HR signals or introduce confounding factors into the HR features, thereby reducing the reliability and accuracy of the HR-based physiologic event detection. Therefore, performing HR sensing or generating HR features in accordance with specified context may help improve the reliability and accuracy of the detection of the target physiologic events.

The heart rate analyzer circuit 210 can sense cardiac activity and perform a plurality of HR feature measurement in response to the context detector 201 detecting one or more contextual conditions indicative or correlative of a change of the patient's metabolic demand, such as an elevated metabolic demand that is indicated by, for example, an increase in body temperature, an increase in respiration rate, or an increase in depth of respiration, among others. In an example, when the context detector 201 detects a time of day during which HR signal is acquired and analyzed, the heart rate analyzer circuit 210 can sense cardiac activity and perform a plurality of HR feature measurements only during specified time of a day, such as afternoon or a period of time excluding night time. In another example, the context detector 201 can detect physical activity or exertion level, or sleep/awake state; and the heart rate analyzer circuit 210 can sense cardiac activity and perform a plurality of HR feature measurements only when the physical activity or exertion level exceeds a threshold, or only when the patient is awake. Examples of the contextual information and the measurements of HR features based on the detected contextual information are discussed below, such as with reference to FIG. 4.

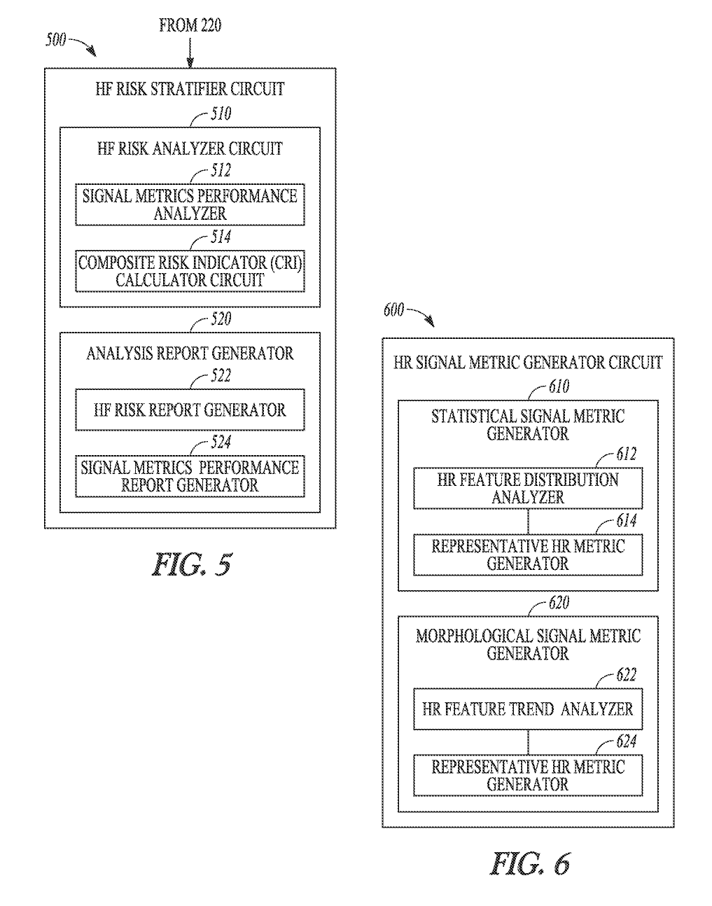

The target event indicator generator circuit 220 can be configured to generate a HR signal metric such as using multiple HR feature measurements. The HR signal metric can be a representative value of the patient's HR under a specified contextual condition. The HR signal metric can include a statistical feature or a morphological feature derived from the plurality of HR feature measurements. Examples of the target event indicator generator circuit 220 are discussed below, such as with reference to FIG. 6.

In some examples, the heart rate trend-based physiologic event detector circuit 200 can optionally include additional physiological signal analyzer circuits configured to produce physiologic signals used for assisting the detection the target physiologic event. For example, physiologic sensors such as a pressure sensor, an impedance sensor, an activity sensor, a temperature sensor, a respiration sensor, or a chemical sensor can be deployed inside or otherwise associated with the patient body. These sensors can each sense one or more physiological signals including thoracic impedance, intracardiac impedance, arterial pressure, pulmonary artery pressure, left atrial pressure, RV pressure, LV coronary pressure, coronary blood temperature, physiologic response to activity, apnea hypopnea index, one or more respiration signals such as a respiration rate signal or a tidal volume signal, or blood oxygen saturation. Physiologic signal features can be generated from respective physiologic signals, and the target event indicator generator circuit 220 can generate a signal metric such as a statistical feature or the morphological feature derived from a plurality of measurements of the corresponding physiologic signal feature.

The physiologic event detector circuit 230 can receive from the target event indicator generator circuit 220 the HR signal metric, and optionally signal metrics of other physiologic signals, and detect a physiologic target event or condition using the signal metrics. A target event or condition can include a physiologic event indicative of an onset of a disease, or a change (e.g., worsening) of a disease state. Examples of target event or condition can include an event indicative of HF decompensation status, change in HF status such as worsening HF, pulmonary edema, or myocardial infarction. In an example, the physiologic event detector circuit 230 can detect a worsening HF event or HF decompensation event if and when the HR signal metric, such as a representative value of patient's HR under a specified contextual condition, exceeds a specified HR threshold. In an example, the HR threshold is approximately 110 beats per minute (bpm). In another example, the HR threshold is approximately within the range of 100-150 bpm.

In some examples, the physiologic event detector circuit 230 can be configured to generate a trend of representative values of the signal metrics over a specified time period, and to detect a target physiologic event using at least the trend of representative values of the signal metrics. In an example, the physiologic event detector circuit 230 can determine the trend by calculating a detection index (DI) representing temporal variation of the values of the signal metrics. For example, the DI can be computed as a difference between a first statistical measure of the signal metric computed from a first time window and a second statistical measure of the signal metric computed from a second time window. The first and the second statistical measures can each include a mean, a median, a mode, a percentile, a quartile, or other measures of central tendency of the signal metric values in the respective time window. In an example, the second time window can be longer than the first window, and at least a portion of the second time window precedes the first time window in time. The second statistical measure can represent a baseline value of the signal metric. In some examples, the signal metrics can include a composite signal metric computed using two or more physiological signals.

The controller circuit 240 can control the operations of the context detector 201, the heart rate analyzer circuit 210, the target event indicator generator circuit 220, the physiologic event detector circuit 230, and the data flow and instructions between these components. The controller circuit 240 can receive external programming input from the instruction receiver circuit 250 to control one or more of detecting physiologic or environmental contextual conditions, sensing of physiologic signals, detecting contextual information, generating signal metrics, or detecting the target physiologic event. Examples of the instructions received by instruction receiver 250 may include: selection of electrodes or sensors used for sensing cardiac activity signals such as the electrograms, extracting HR features, or the configuration of the target event detection. The instruction receiver circuit 250 can include a user interface configured to present programming options to the user and receive user's programming input. In an example, at least a portion of the instruction receiver circuit 250, such as the user interface, can be implemented in the external system 120.

FIG. 3 illustrates an example of a heart rate-based heart failure risk stratifier circuit 300, which can be an embodiment of the heart rate-based HF event heart detection/risk assessment circuit 113. The heart rate-based heart failure risk stratifier circuit 300 can include one or more of a context detector 201, a heart rate analyzer circuit 210, a target event indicator generator circuit 220, a heart failure (HF) risk stratifier circuit 330, a controller circuit 340, and an instruction receiver circuit 350.

As discussed in the heart rate trend-based physiologic event detector circuit 200 with reference to FIG. 2, the context detector 201 can be configured to detect contextual condition associated with a patient, including a patient's physiologic context and an environmental context. The heart rate analyzer circuit 210 can sense a cardiac activity signal, generate one or more HR features, and perform a plurality of HR feature measurements in response to the detected contextual conditions meeting a specified condition, such as indicating an elevated metabolic demand. The heart rate analyzer circuit 210 can include a cardiac activity sensor circuit 212 configured to sense from a patient a cardiac electrical or cardiac mechanical signal such as by using implantable, wearable, or otherwise ambulatory electrodes or physiologic sensors. The cardiac activity sensor circuit 212 can detect from the sensed cardiac signals electrophysiological events or the detected mechano-physiologic events. The HR feature generator circuit 214 can generate one or more HR features (including, for example, HR, CL, or a variability of HR or a variability of CL) using the detected electrophysiological events or the detected mechano-physiologic events. The target event indicator generator circuit 220 can generate a HR signal metric such as using a plurality of HR feature measurements obtained when the contextual condition meets a specified criterion. The HR signal metric can be a representative value of patient's HR under the specified contextual condition. The HR signal metric can include a statistical feature or a morphological feature derived from the plurality of HR feature measurements. Examples of the target event indicator generator circuit 220 are discussed below, such as with reference to FIG. 6.

The heart failure (HF) risk stratifier circuit 330 can receive input from the target event indicator generator circuit 220, and calculate a composite risk indicator (CRI) using at least the HR signal metric such as produced by the target event indicator generator circuit 220. The CRI can indicate the likelihood of the patient developing a future event indicative of worsening HF, such as developing a figure HF decompensation event in a specified timeframe, such as within approximately 1-3 months, 3-6 months, or beyond 6 months. The HF risk stratifier circuit 330 can also be used to identify patients at elevated risk of developing a new disease or worsening an existing disease, such as pulmonary edema, pulmonary condition exacerbation such as COPD, asthma and pneumonia, myocardial infarction, dilated cardiomyopathy (DCM), ischemic cardiomyopathy, systolic HF, diastolic HF, valvular disease, renal disease, chronic obstructive pulmonary disease (COPD), peripheral vascular disease, cerebrovascular disease, hepatic disease, diabetes, asthma, anemia, depression, pulmonary hypertension, sleep disordered breathing, or hyperlipidemia, among others.

The HF risk stratifier circuit 330 can generate two or more categorical risk levels using a comparison between the CRI and a reference measure. The categorical risk levels can indicate elevated risk of the patient developing a future event indicative of worsening heart failure. For example, the categorical levels can include “high risk”, “medium risk”, or “low risk.” A higher degree of dissimilarity between the CRI and the reference can indicate a higher risk of the patient developing HF events in the future than an average patient with the similar chronic conditions.

The controller circuit 340 can control the operations of the context detector 201, the heart rate analyzer circuit 210, the target event indicator generator circuit 220, the HF risk stratifier circuit 330, and the data flow and instructions between these components. Similar to the controller circuit 240 with reference to FIG. 2, the controller circuit 340 can receive external programming input from the instruction receiver circuit 350 to control one or more of detecting physiologic or environmental contextual conditions, sensing of physiologic signals, detecting contextual information, generating signal metrics, or calculating the CRI. The instruction receiver circuit 350 can include a user interface configured to present programming options to the user and receive user's programming input. In an example, at least a portion of the instruction receiver circuit 350, such as the user interface, can be implemented in the external system 120.

FIG. 4 illustrates an example of a context receiver circuit 400, which can be an embodiment of the context detector 201. The context receiver circuit 400 can be configured to generate at least one contextual condition associated with a patient. When communicatively connected to the heart rate analyzer circuit 210, the context receiver circuit 400 can trigger a HR feature measurement session such that the heart rate analyzer circuit 210 can perform a plurality of HR feature measurements in response to the at least one contextual condition meeting a specified criterion. The context receiver circuit 400 can also control the target event indicator generator circuit 220 to generate the HR signal metric using a portion of the HR feature measurements, such as the measurements if and when at least one contextual condition meets a specified criterion.

The context receiver circuit 400 can include at least one of an environmental context detector 410 and a physiologic context detector 420. The environmental context detector 410 and the physiologic context detector 420 each can be coupled to a sensor that can sense a physical or physiological condition associated with the patient. In some examples, the environmental context detector 410 and the physiologic context detector 420 each can access a machine-readable medium that stores the environmental context information or patient's medical record, or receive environmental context or the patient physiologic responses from an end-user such as via a user-interface connected to the instruction receiver circuit 250.

The environmental context detector 410 can be coupled a sensor that can sense a physical parameter indicative of the patient's ambient environmental condition external to the patient, but can likely affect the patient's health or disease states. The environmental context detector 410 can include one or more of a timer/clock circuit 411, an ambient temperature receiver 412, and a barometric pressure receiver 413.

The timer/clock circuit 411 can be capable of determining time of a day, such as morning, afternoon, or evening of the day. The heart rate analyzer circuit 210 can perform a plurality of HR feature measurements non-selectively during 24 hours a day, or selectively during a specified time of day such as when the patient is anticipated to have an elevated metabolic demand higher than other time of the day. In an example, the analyzer circuit 210 can measure HR during the morning hours such as from 6 a.m. to 12 p.m. In another example, the HR analyzer circuit 210 can measure HR during the afternoon hours such as from 12 p.m. to 6 p.m. In another example, the heart rate analyzer circuit 210 can measure HR during the period of the day excluding the night time (e.g., midnight to 6 a.m.).

The ambient temperature receiver 412 can receive values of the ambient temperature such as from a thermometer. The barometric pressure receiver 413 can receive values of the barometric pressure such as from a barometer. The ambient temperature receiver 412 or the barometric pressure receiver 413 can also respectfully receive the ambient temperature values or the barometric pressure values from an end-user such as via the user-interface connected to the instruction receiver circuit 250. The ambient temperature or the barometric pressure may impact the patient's physiology and cause variations in signal metrics used for HF event detection or for HF risk stratification. For example, high environmental temperature or low barometric pressure can increase the metabolic demand of the patient, resulting in one or more presentations including an increase in heart rate, an increase in respiration rate or depth of respiration, an increase in body temperature, among other physiologic responses. With the elevated metabolic demand, underlying HF disease or worsening HF status can be more likely triggered and manifested in the HR signal metric. The ambient temperature receiver 412 or the barometric pressure receiver 413 can trigger a HR feature measurement session, causing the HR feature generation circuit 214 to perform a plurality of HR feature measurements when the ambient temperature or the barometric pressure meet respective criterion such as correlative of an increase in the patient's metabolic demand.

The physiologic context detector 420 can be coupled to one or more sensors that can sense a parameter indicating patient's physiological or psychological conditions. The physiologic context detector 420 can include one or more of a metabolic demand detector 421, an anxiety level detector 422, a posture or activity detector 423, and a sleep/awake state detector 424. The metabolic demand detector 421 can be configured to detect the patient's metabolic demand, or the change of the metabolic demand. The metabolic demand detector 421 can be coupled to one or more physiologic sensors, including a respiration sensor that can sense the change in respiration rate or tidal volume, a body temperature sensor that can sense a change in body temperature, or a chemical sensor that can sense oxygen or carbon dioxide level in the body. In an example, the metabolic demand detector 421 can detect a change in patient's metabolic demand when the one or more physiologic sensors produce information correlative of an increase in patient's metabolic demand, such as an increase in heart rate, an increase in respiration rate or depth of the respiration, or an increase in body temperature. The heart rate analyzer circuit 210, coupled to the context receiver circuit 400, can perform multiple HR feature measurements in response to detection of one or more of an increase in body temperature, an increase in respiration rate, or an increase in depth of respiration.

The anxiety level detector 422 can detect an indication of the patient's anxiety or stress level during a specified period. The anxiety level detector 422 can be coupled to a physiologic sensor configured to detect an indication of patient stress level, or receive input about patient's stress level from an end-user input via the user-interface connected to the instruction receiver circuit 250. The heart rate analyzer circuit 210 can perform multiple HR feature measurements in response to a detection of a stress level indicative or correlative of an increase in metabolic demand, such as when the detected stress level is above a specified threshold.

The posture or activity level detector 423 can be coupled a sensor such as an accelerometer and detect a posture of the patient as being one of two or more posture states including, for example, a supine or upright position. The posture or activity level sensor 423 can also detect patient's strenuousness of the activity. The heart rate analyzer circuit 210 can perform multiple HR feature measurements in response to the detected posture being classified as a specified state indicative or correlative of the elevated metabolic demand, such as an upright posture. In an example, the heart rate analyzer circuit 210 can perform multiple HR feature measurements in response to a detection of an increase in the metabolic demand (such as produced by the metabolic demand detector 421 or the anxiety level detector 422) and the detected activity or exertion level below a specified threshold (such as reduced frequency, time, or vigorousness of activity over a given time period).

The sleep/awake state detector 424 can be configured to receive an indication of a change from a steep state to an awake state, such as using a sleep detector. Examples of the sleep detector can include accelerometers, piezoelectric sensor, biopotential electrodes and sensors, or other physiologic sensors configurable to detect the posture, change of posture, activity, respiration, electroencephalograms, or other physiologic signals indicative of sleep or awake states. The sleep/awake state detector 424 can also receive indications of a sleep-to-awake state transition from an end-user such as via a user-interface connected to the instruction receiver circuit 250. In an example, the received transition from a sleep state to an awake state can be used to trigger a HR feature measurement sessions such that the HR feature generation circuit 214 can perform a plurality of HR feature measurements upon the detection of a transition from a sleep to awake state.

FIG. 5 illustrates an example of a HF risk stratifier circuit 500, which can be an embodiment of the HF risk stratifier circuit 330. The HF risk stratifier circuit 500 can include HF risk analyzer circuit 510 and an analysis report generator 520. The HF risk stratifier circuit 500 can receive one or more signal metrics including the HR signal metric from the target event indicator generator circuit 220, analyze the signal metrics, and determine a quantity such as a composite risk indicator (CRI) indicative of the likelihood of the patient later developing a target physiologic event such as an HF decompensation event.

The HF risk analyzer circuit 510 can include a signal metrics performance analyzer 512 and a composite risk indicator (CRI) calculator circuit 514. The signal metrics performance analyzer 512 can be configured to generate for each of one or more of the signal metrics a respective performance measure that indicates reliability or accuracy of detecting a target physiologic event such as an HF decompensation event, or identifying patient at a higher risk of experiencing an HF decompensation event. Examples of the performance measures can include a predicted hazard ratio (R_{H}), a predicted likelihood of the patient later developing a target physiologic event, a predicted sensitivity (Se), a predicted specificity (Sp), a positive predictive value (PPV), a negative predictive value (NPV), or a predicted signal quality (Sq), each of which can be determined using population-based statistics.

The signal metrics performance analyzer 512 can determine the predicted signal quality of a signal metric such as HR. Examples of the signal quality can include signal strength, signal variability, or signal-to-noise ratio, among others. Signal variability can include range, inter-quartile range, standard deviation, variance, sample variance, or other first-order, second-order, or higher-order statistics representing the degree of variation. For example, in determining the quality of the HR signal metric, the signal metrics generator circuit 320 can produce a plurality of HR feature measurements such as during a specified period of time. The signal metrics performance analyzer 512 can determine the variability of the HR feature measurements such as by computing a variance of the HR measurements. A high signal quality, such as indicated by one or more of high signal-to-noise ratio, high signal strength, or low signal variability, is desirable for identifying patients at an elevated risk of developing future HF events.

The composite risk indicator (CRI) calculator circuit 514 can generate a CRI using one or more signal metrics. In an example, the signal metrics performance analyzer 512 can calculate for each signal metrics (Mᵢ) a respective individual risk score (R_{Mi}) using a probability model (f) and one or more of the predicted hazard ratio (R_{H}), the predicted sensitivity (Se), the predicted specificity (Sp), the positive predictive value (PPV), the negative predictive value (NPV), and the predicted signal quality (Sq). That is, R_{Mi}=f(R_{H}, Se, Sp, PPV, NPV, Sq). The CRI calculator circuit 514 can compute the CRI using a linear or nonlinear combination of the risk scores (R_{Mi}) associated with respective signal metrics. The CRI can be computed as weighted sum of the risk scores, where each risk score can be scaled by a respective weight factor proportional to a performance measure of the signal metric. The CRI can also be determined as a parametric or non-parametric model using the individual risk scores, such as decision trees, neural network, Bayesian network, among other machine learning methods. In another example, the CRI calculator circuit 514 can generate a CRI using a linear or nonlinear combination of signal metrics that have performance measures above a specified threshold. The CRI can be computed as a function of weighted sum of the signal metrics, where each signal metric can be scaled by a respective weight factor proportional to a performance measure of the signal metric. The CRI can also be determined as a parametric or non-parametric model using the individual signal metric, such as decision trees, neural network, Bayesian network, among other machine learning methods.

The analysis report generator 520 can include HF risk report generator 522 and a signal metrics performance report generator 524. The HF risk report generator 522 can generate a report to inform, warn, or alert a system end-user an elevated risk of a patient developing a future HF event. The report can include the CRI with corresponding timeframe within which the risk is predicted. The report can also include recommended actions such as confirmative testing, diagnosis, or therapy options. The report can include one or more media formats including, for example, a textual or graphical message, a sound, an image, or a combination thereof. In an example, the HF risk report generator 522 can be coupled to the instruction receiver circuit 250 and the report can be presented to the user via an interactive user interface on the instruction receiver circuit 250. The HF risk report generator 402 can be coupled to the external device 120, and be configured to present to the user the risk (e.g., the CRI) of patient developing future HF events via the external device 120.

The signal metrics performance report generator 524 can generate, and present to the user, one or more of a report including the contextual conditions such as detected by the context detector 201, the cardiac activity signals such as received by the cardiac activity sensor circuit 212, and the signal metrics including the HR signal metric such as generated by the target event indicator generator circuit 220. The signal metrics performance report generator 524 can be coupled to the external device 120 or the instruction receiver circuit 250, and be configured to present the signal metrics information to the user therein. The user input can include confirmation, storage, or other programming instructions to operate on the signal metrics.

FIG. 6 illustrates an example of a HR signal metric generator circuit 600, which can be an embodiment of the target event indicator generator circuit 220. The HR signal metric can be a representative value of patient's HR under a specified contextual condition. The HR signal metric generator circuit 600 can include one or both of a statistical signal metric generator 610 and a morphological signal metric generator 620.

The statistical signal metric generator 610 can be configure to generate a statistical parameter from the plurality of HR feature measurements such as provided by the HR analyzer circuit 210 when a contextual condition meets a specified criterion. Examples of the statistical parameter can include mean, median, or other central tendency measures, standard deviation, variance, correlation, covariance, or other higher-order statistics computed from the plurality of HR feature measurements, among others. The statistical parameter of the HR feature measurements can also be determined using a statistical distribution of the the plurality of HR feature measurements. As illustrated in FIG. 6, the statistical signal metric generator 610 can include a HR feature distribution analyzer 612 and a representative HR metric generator 614. The HR feature distribution analyzer 612 can generate a histogram of the plurality of HR feature measurements. In another example, the HR feature distribution analyzer 612 can further approximate the histogram of the HR feature measurement by a statistical distribution function. The histogram or the approximated statistical distribution function each indicates the frequency of occurrence of a HR feature value during the period when the plurality of HR feature measurements are taken.

The representative HR metric generator 614 can determine a representative HR (HR_{Rep}) using the plurality of HR feature measurements and a threshold value associated with the statistical distribution or histogram of the HR feature measurements. An example of such a threshold value is a percentile rank (PR) that indicates relative number of HR measurements (e.g., percentage of the plurality of the HR feature measurements) with values falling below or equal to the HR_{Rep}. For example, a 25-th percentile taken from N HR feature measurements {HR₁, HR₂, . . . , HR_{N}} corresponds to a HR_{Rep} where 25% of the HR measurements {HR₁, HR₂, . . . , HR_{N}} are less than or equal to HR_{Rep}.

A representative HR metric value HR_{Rep} associated with a PR less than 50-th percentile (such as 15-th percentile) corresponds to lower HR value among the plurality of HR feature measurements. Conversely, HR_{Rep} associated with a PR higher than 50-th percentile (such as 80-th percentile) corresponds to higher HR value among the plurality of HR feature measurements. A higher HR value can happen when the patient is awake, physically active, or has an elevated metabolic demand. The present inventors have recognized that a HR feature measured under these contextual conditions, which is associated with a PR greater than 50-th percentile out of a plurality of HR feature measurements, can be both sensitive and specific in detecting a HF decompensation event, or in predicting a likelihood that patient develops a future HF decompensation event. In an example, the PR can be approximately 85-th percentile.

The representative HR metric generator 614 can receive a specified PR from an end-user such as via the instruction receiver circuit 250, or alternatively from a data storage unit such as a memory circuit where a pre-determined PR is stored. In an example, the specified PR is greater than 50%, such that representative HR metric value HR_{Rep} represents a HR of K-th percentile (K>50) of the plurality HR measurements. That is, K% of the HR measurements provided by the HR analyzer circuit 210 are less than or equal to the representative HR.

The morphological signal metric generator 620 can include a HR feature trend analyzer 622 and a representative HR metric generator 624. The feature trend analyzer 622 can be configured to create a HR feature trend using a plurality of HR feature measurements. A HR feature trend can be a time-series signal representing temporal variation of the heart rate. The representative HR metric generator 624 can generate one or more morphological features from a HR feature trend signal. Examples of the morphological parameters can include maximum or minimum within a specified period, amount of change within a specified period, positive or negative slope that indicates the rate of increase or rate of decrease, signal power spectral density at a specified frequency range, among other morphological descriptors.

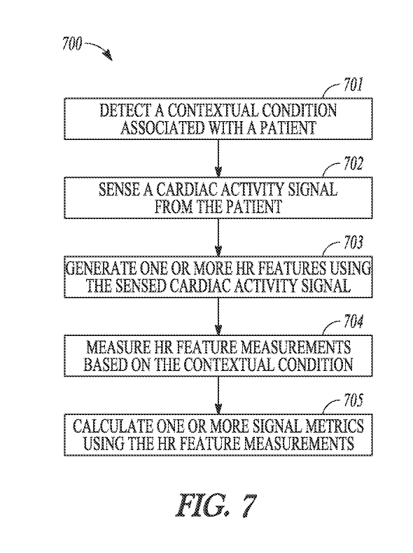

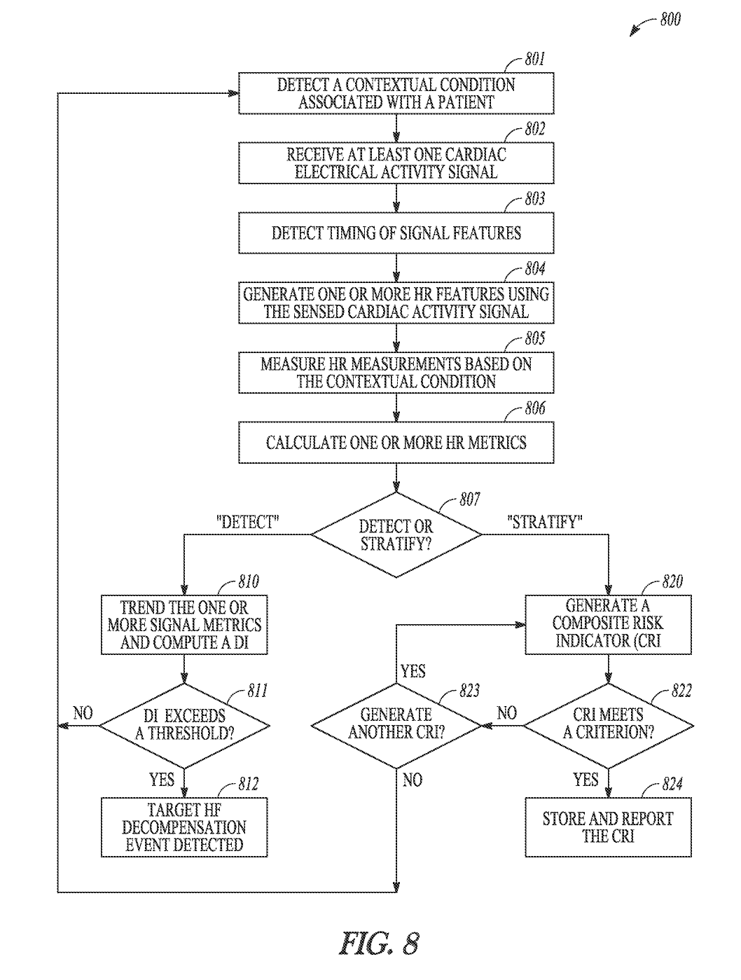

FIG. 7 illustrates an example of a method 700 for evaluating heart rate (HR) under specified contextual conditions. The method 700 can generate one or more signal metrics including a representative HR metric which can be used for detecting a target physiologic event such as worsening HF, or for predicting a likelihood of a patient developing a future target physiologic event. The method 700 can be implemented and operate in an ambulatory medical device or in a remote patient management system. In an example, the method 700 can be performed by the heart rate-based HF event detection/risk assessment circuit 113 implemented in the IMD 110, or the external device 120 which can be in communication with the IMD 110.

At 701, a contextual condition associated with a patient can be detected. The contextual condition can include at least one of a patient's physiologic context or an environmental context. The physiologic context can include body-related contextual information, such as a posture, a physical activity or exertion level, a sleep or awake state, a mental or emotional state, metabolic demand, body temperature, and other parameters indicative of patient health status or body conditions. The environmental context can include factors external to the patient but likely to affect patient's health or disease states, such as the ambient temperature, barometric pressure, humidity, social environment, among others. The physiologic context or the environment context can correlate to the patient's elevated metabolic demand. The contextual condition can be sensed using a physical sensor or a physiologic sensor. For example, a timer/clock circuit can be used to provide contextual condition of time of a day, a thermometer can provide ambient temperature, a body temperature sensor can provide an indication of a change of the patient's metabolic demand, an accelerometer can provide information regarding one or more of patient's posture, physical activity or exertion level, or indication of the sleep/awake state. Alternatively or additionally, the contextual condition can be obtained via a user-input such as via the user-interface connected to the instruction receiver circuit 250.

At 702, a cardiac activity signal can be sensed from the patient such as by using or more implantable, wearable, or otherwise ambulatory cardiac activity sensor each configured to sense from a patient a cardiac activity signal. The cardiac activity signal can be a cardiac electrical signal or a cardiac mechanical signal. Examples of the cardiac electrical signal can include an electrocardiogram (ECG), a subcutaneous electrocardiogram (ECG) such as sensed by electrodes placed subcutaneously (e.g., under the skin), or an electrogram (EGM) sensed from inside the heart chamber, at or within the heart tissue, or on or near the surface of the heart. Examples of the cardiac mechanical signal can include a heart sound signal such as sensed by an accelerometer or a microphone, intracardiac impedance signal that varies as a result of cyclic cardiac contractions, or an arterial pulse signal indicative of pulsatile blood pressure or blood flow caused by cyclic contraction of the heart and the opening/closure of aortic or pulmonic valves.

At 703, the cardiac activity signal can be processed, including amplification, digitization, filtering, or other signal conditioning operations. From the processed cardiac electrical signal, one or more electrophysiological events such as a P wave, an R wave, a T wave, a QRS complex, or other ECG components representing depolarization, hyperpolarization, repolarization, or other electrophysiological properties of the myocardium can be extracted. Alternatively or additionally, one or more mechano-physiologic events can be extracted from cardiac mechanical signals, including S1, S2, S3, or S4 heart sound from the sensed heart sound signal, peak or trough impedance from the cardiac impedance signal, or peak or trough blood pressure from the blood pressure signal, among others. One or more HR features can be generated using the detected electrophysiological events or the detected mechano-physiologic events. Examples of HR feature can include HR or cycle length (CL), or a variability of HR or a variability of CL.

At 704, a plurality of measurements of the HR features can be obtained in response to the detected patient contextual condition meeting a specified criterion. In an example where the detected contextual condition includes a timer/clock, a plurality of HR feature measurements can be measured during specified time of a day, such as during afternoon or a period of time excluding night time. In another example where the detected contextual condition includes the patient's sleep or awake state, the measurements of HR feature can be taken only when the patient is awake. In an example where the detected contextual condition includes the patient's physical activity or exertion level, the measurements of HR feature can be taken only when the patient's physical activity or exertion level exceeds a specified threshold. In yet another example where the detected contextual condition includes an indication of patient's metabolic demand, the multiple measurements of HR feature can be performed only if the metabolic demand exceeds a specified level, such as when the patient respiration rate or the body temperature exceeds a respective threshold.

At 705, one or more signal metrics, including a HR metric, can be calculated using at least the plurality of HR feature measurements. The HR metric can be a representative value of patient's HR under a specified contextual condition. The HR signal metric can include a statistical parameter or a morphological parameter derived from the plurality of HR feature measurements. Examples of the statistical parameter can include mean, median, or other central tendency measures, standard deviation, variance, correlation, covariance, or other higher-order statistics computed from the plurality of HR feature measurements, among others. In an example, a histogram or a statistical distribution function can be generated using the plurality of HR feature measurements, and the HR signal metric, or a representative HR (HR_{Rep}), can be computed using the plurality of HR feature measurements and a threshold value associated with the statistical distribution or histogram of the HR feature measurements. In an example, the threshold value is a percentile rank (PR) that indicates relative number of HR measurements (e.g., percentage of the plurality of the HR feature measurements) with values falling below or equal to the HR_{Rep}. In an example, the specified PR is greater than 50%, such that representative HR metric value HRRep represents a HR of K-th percentile (K>50) of the plurality HR measurements.

In some examples, the signal metrics can be composite signal metrics generated from two or more physiological signals. The signal metrics can be presented to the end-user for monitoring the patient health status or disease progress such as worsening HF. The signal metrics can also be used for detecting the presence of a target physiologic event such as an indication of an HF decompensation event, for predicting the future risk of developing a target physiologic event, or for titrating medical or device therapies to the patient such as by adjusting the dosage or parameters associated with the electrical stimulation.

FIG. 8 illustrates an example of a method 800 for detecting an HF event indicative of HF decompensation or providing a risk stratification of a future HF event. The method 800 can be an embodiment of the method 700 for evaluating HR under specific contextual conditions, further including method for detecting a present HF event or predicting a future HF event using at least a HR metric generated under specified contextual conditions. In an example, the method 800 can be performed by the HR-based HF event detection/risk stratification circuit 113.

At 801, a contextual condition, including one or more of a patient's physiologic contexts or environmental contexts associated with the patient, can be detected such as using one or more physical sensors or physiologic sensors. Examples of the contextual condition can include a time of day or, patient physical activity or exertion level, patient sleep/awake state, or an indication of patient metabolic demand, among others. At 802, at least one cardiac electrical activity signal can be received from a patient. The cardiac electrical activity signal can include intracardiac electrogram (EGM), surface electrocardiogram (ECG), subcutaneous ECG, or any other cardiac signals indicative of electrical activity of a heart. The cardiac electrical activity signal can be sensed using a physiologic sensor or a plurality of electrodes attached to or implanted within the patient body, such as two or more electrodes on one or more of transvenous leads 108A-C coupled to an implantable medical device (IMD) and the can 112 of the IMD. The received cardiac electrogram can be processed at 803 to generate one or more signal features including a P wave, an R wave, a T wave, a QRS complex, or other components representing depolarization, hyperpolarization, repolarization, or other electrophysiological properties of the myocardium. Timing information of these signals features can also be determined. At 804, one or more HR features, including HR, cardiac cycle length (CL), or a variability of HR or a variability of CL, can be generated using the timing of the signals features.

A plurality of measurements of the one or more HR features can be measured at 805 in response to the detected patient contextual condition meeting a specified criterion. In various examples, the HR feature measurement can be performed only during a specified time of day such as afternoon; when the patient is awake; when the body temperature, the respiration rate, or the stress level is within a specified range; or when the air temperature or the barometric pressure in the patient's ambient environment is within a specified range.

One or more signal metrics, including a HR metric, can be calculated at 806 using at least the plurality of HR feature measurements. The HR metric can be a representative value of patient's HR under a specified contextual condition. The HR signal metric can include a statistical parameter or a morphological parameter derived from the plurality of HR feature measurements.

At 807, a decision is made, such as by an end-user through a programming device, to select either to detect the presence of an event indicative of HF decompensation, or to stratify patient's risk of developing a future event indicative of HF decompensation. If the choice is to “detect” HF decompensation, then at 810, a trend of can be generated using the one or more signal metrics, such as a trend of daily HR metric that indicates temporal variation of the daily HR metric over a period of approximately 1-3 months, 3-6 months, or beyond 6 months. The trend can be quantitatively represented by a detection index (DI). In one example, the DI can be a statistical measure of the HR metric within a specified time window of approximately 14-28 days. Examples of the statistical measure can include a mean, a median, a mode, a percentile, a quartile, or other measures of central tendency of the signal metric values. In another example, the DI can be calculated as a difference between a first statistical measure of the signal metric computed from a first time window and a second statistical measure of the signal metric computed from a second time window. The second time window can be longer than the first window, and at least a portion of the second time window precedes the first time window in time. The second statistical measure can be indicative of a baseline value. For example, a DI from the trend of HR metric can be the difference between an average HR metric in the first window of approximately 14-28 days and an average HR metric in the second window of approximately 1-6 months preceding the first window. The DI thus computed indicates the relative change of HR metric over a baseline.

A decision is made at 811 as to whether the DI meets a specified criterion, such as exceeding a specified threshold. The target HF decompensation event is deemed detected at 812 if, for example, the HR metric exceeds a HR threshold, or if the difference from the baseline HR metric exceeds a specified. threshold. If the DI does not meet the criterion, then no target HF event is deemed detected, and the patient monitoring can be continued with detecting contextual condition at 801.

If at 807 the choice is made to “stratify” the patient's risk, then at 820 a composite risk indicator (CRI) can be generated. The CRI can be a quantity that indicates the likelihood of the patient developing a future event indicative of worsening HF, such as excessive intrathoracic fluid accumulation, increased heart sounds, increased heart rate, increased respiratory rate, decreased tidal volume, reduction in physical activity, or other events indicative of HF decompensation status. The CRI can be calculated using one or more signal metrics including the HR metrics. When multiple signal metrics in addition to the HR metric are used for risk stratification of a future HF event, the CRI can be computed as a linear or nonlinear combination of individual risk scores associated with respective signal metrics. The individual risk score for a signal metric can be determined based on the deviation of the value of the signal metric from a specified reference value. For example in assigning a score for the HR metric determined at 806, a higher score can be assigned if the HR metric value is far greater than a reference or threshold value such as 110 bpm, and a lower score can be assigned if the HR metric value is close to or lower than the reference value. The CRI can also be determined as a parametric or non-parametric model using the individual risk scores, such as decision trees, neural network, Bayesian network, among other machine learning methods.

At 822, the CRI can be compared against a specified criterion, such as a threshold value, to determine the risk of the patient developing a future HF event. The threshold value can be computed from data from a patient population, which indicates an “average” patient's risk of developing future HF events. The reference measures can include: the mean, median, a range, or other central tendency of the risk across the patient population; variance, standard deviation, or other second or higher order statistical measures across the patient population; histogram, statistical distribution, or the parametric or non-parametric model representing the histogram or statistical distributions.

Based on the comparison between the CRI and the threshold value, the CRI can be categorized to two or more categorical risk levels indicating elevated risk of the patient later developing the HF event. For example, the categorical levels can include “high risk”, “medium risk”, or “low risk.” A higher degree of dissimilarity between the CRI and the reference can indicate a higher risk of the patient developing HF events in the future than an average patient with the similar chronic conditions.

If at 822 the CRI meets the specified criterion such as the CRI value being categorized as “medium risk” or “high risk” level, then at 824 a report is generated to inform, warn, or alert the end-user the elevated risk of the patient developing a future HF event. The report can include any or all of the information of the signal metrics selected for analysis, the CRI, the categorical classifications of CRI, one or more composite risk indices with corresponding timeframe within which the risk is predicted. The report can also include recommendations for intervention, further testing, or treatment options for the patient. The report can be in a form of a textual or graphical message, a sound, an image, or any combination thereof.

If the CRI does not meet the specified criterion, then at 823 a decision is made as to whether a new CRI is to be computed such as using additional signal metrics. The decision at 823 can be received from an end-user such as using a programming device, or automatically executed in response to the CRI failing to meet the criterion by a narrow margin. If additional signal metrics are decided to be used at 823, then a new CRI can be generated at 821. Otherwise, the patient is deemed at low risk of developing a future HF event, and no preventive action is deemed necessary. The patient monitoring can be continued with receiving the physiological signals such as the cardiac electrogram at 801.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system, comprising:
a context detector circuit configured to detect contextual condition associated with a patient, the contextual condition including information indicative or correlative of a change in metabolic demand of a patient;
a heart rate (HR) analyzer circuit configured to sense from the patient a cardiac activity signal, extract a HR feature from the sensed cardiac activity signal, and perform a plurality of measurements of the HR feature in response to the detected patient contextual condition meeting a specified criterion;
a target event indicator generator circuit configured to calculate one or more signal metrics using the plurality of measurements of the HR feature; and
a physiologic event detector circuit coupled to the target event indicator generator circuit, the physiologic event detector circuit configured to detect a target physiologic event using the one or more signal metrics.

2. The system of claim 1, wherein:
the target event indicator generator circuit is configured to calculate the one or more signal metrics including a statistical or morphological parameter extracted from the plurality of measurements of the HR feature; and
the physiologic event detector circuit is configured to detect the worsening heart failure (HF) using the one or more signal metrics.

3. The system of claim 2, wherein the target event indicator generator circuit is configured to calculate the one or more signal metrics including a central tendency of the plurality of measurements of the HR feature.

4. The system of claim 2, wherein the target event indicator generator circuit is configured to calculate the one or more signal metrics including a representative HR (HR_{Rep}) representing a specified percentile rank among the plurality of measurements of the HR feature, the specified percentile rank indicative of a relative number of HR measurements falling below or equal to the HR_{Rep}.

5. The system of claim 4, wherein the specified percentile rank includes a HR percentile greater than 50-th percentile.

6. The system of claim 1, wherein the context detector circuit includes a timer/clock circuit capable of determining time of a day, and wherein the HR analyzer circuit is configured to perform a plurality of measurements of the HR feature during specified time of a day indicative or correlative of an elevated metabolic demand of the patient.

7. The system of claim 6, wherein the HR analyzer circuit is configured to perform a plurality of measurements of the HR feature during a period of time excluding night of the day.

8. The system of claim 1, wherein the context detector circuit includes a sleep state detector configured to detect in the patient a time of transition from a sleep state to an awake state, and wherein the HR analyzer circuit is configured to perform a plurality of measurements of the HR feature in response to the detected transition from the sleep state to the awake state.

9. The system of claim 1, wherein the context detector circuit includes a posture sensor configured to detect a posture of the patient and to classify the posture as one of two or more posture states, and wherein the HR analyzer circuit is configured to perform a plurality of measurements of the HR feature in response to the detected posture being classified as a specified state indicative or correlative of an elevated metabolic demand.

10. The system of claim 1, wherein the context detector circuit includes one or more physiologic sensors configured to detect a change in metabolic demand of the patient during a specified period, and wherein the HR analyzer circuit is configured to perform a plurality of measurements of the HR feature in response to a detection of an increase in the metabolic demand.

11. The system of claim 1, wherein the context detector circuit includes an activity sensor configured to detect a physical activity or exertion level of the patient, wherein the HR analyzer circuit is configured to perform a plurality of measurements of the HR feature in response to the detected physical activity or exertion level exceeding a specified activity level threshold.

12. A system, comprising:
a signal analyzer circuit, including:
a context detector circuit configured to receive contextual condition associated with a patient, the contextual condition including time of a day or a physical activity or exertion level of the patient;
a HR analyzer circuit configured to receive a cardiac activity signal from the patient, extract a HR feature from the sensed cardiac activity signal, and perform a plurality of measurements of the HR feature in response to the received time of day meets a specified criterion or the received physical activity or exertion level exceeds a specified threshold value; and
a signal metrics generator circuit configured to calculate one or more signal metrics using the plurality of measurements of the HR feature; and
a risk stratifier circuit configured to generate a composite risk indicator (CRI) using the one or more signal metrics, the CRI indicative of the likelihood of the patient developing a future event indicative of a new disease or worsening an existing disease.

13. The system of claim 12, wherein the risk stratifier circuit is configured to generate two or more categorical risk levels using a comparison between the composite risk indicator and a reference measure, the two or more categorical risk levels indicative of elevated risk of the patient developing a future event indicative of worsening heart failure.

14. A method, comprising:
detecting a contextual condition associated with a patient, the contextual condition including information indicative or correlative of elevated metabolic demand of a patient;
sensing from the patient a cardiac activity signal and extracting a HR feature from the sensed cardiac activity signal;
measuring a plurality of measurements of the HR feature in response to the detected patient contextual condition meeting a specified criterion; and
calculating one or more signal metrics using the plurality of measurements of the HR feature.

15. The method of claim 14, wherein calculating the one or more signal metrics includes calculating a statistical or morphological parameter extracted from the plurality of measurements of the HR feature.

16. The system of claim 14, wherein calculating the one or more signal metrics includes calculating a representative HR (HR_{Rep}) representing an n-th percentile rank indicative of a relative amount of HR measurements among the plurality of measurements falling below or equal to the HR_{Rep}, the n-th percentile rank higher than 50-th percentile.

17. The method of claim 14, wherein detecting the contextual condition includes detecting time of a day, and wherein measuring the plurality of HR measurements includes measuring a plurality of measurements of the HR feature during specified time of a day indicative or correlative of an elevated metabolic demand of the patient.

18. The method of claim 14, wherein detecting the contextual condition includes detecting a physical activity or exertion level of the patient, and wherein measuring the plurality of HR measurements includes measuring a plurality of measurements of the HR feature in response to the detected physical activity or exertion level exceeding a specified activity level threshold.

19. The method of claim 14, wherein detecting the contextual condition includes, using one or more physiologic sensors, detecting at least one of a time of transition from a sleep state to an awake state, an increase in body temperature, an increase in heart rate, an increase in pressure, a decrease in physical activity or exertion level, or an increase in respiration rate.

20. The method of claim 14, further comprising at least one of:
detecting a target physiologic event indicative of worsening heart failure using the one or more signal metrics; or
generating a composite risk indicator using the selected one or more signal metrics and classifying the patient into one of two or more categorical risk levels, the composite risk indicator indicative of the likelihood of the patient developing a future event indicative of worsening heart failure.
